(19) United States
(12) Patent Application Publication
(10) Pub. No.: US 2008/0004564 A1
(43) Pub. Date: Jan. 3, 2008

(54) CONTROLLED RELEASE MEMBRANE AND METHODS OF USE

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Tokyo (JP)

(21) Appl. No.: 11/694,621

(22) Filed: Mar. 30, 2007

Related U.S. Application Data
(60) Provisional application No. 60/787,725, filed on Mar. 30, 2006.

(51) Int. Cl. A61N 1/30

(52) U.S. Cl. 604/20

(57) ABSTRACT

This disclosure includes a device comprising a controlled release membrane comprising an electroactive polymer as well as methods of use thereof. In some particular embodiments, the electroactive polymer membrane is utilized in an active agent delivery device comprising a passive or active transport mechanism, including iontophoresis. In certain aspects, an iontophoresis device may be used which includes an active electrode assembly having an active agent solution holding portion; and a non-active electrode assembly. In certain aspects, the electroactive polymer membrane may be cycled from neutral state to charged state, thereby facilitating the administration of the active agent or pharmaceutical drug.

### CROSS-REFERENCE TO RELATED APPLICATION

This application claims the benefit under 35 U.S.C. §119(e) of U.S. Provisional Patent Application No. 60/787,725 filed Mar. 30, 2006, the contents of which are incorporated herein by reference in their entirety.

### BACKGROUND

1. Field

This disclosure generally relates to the field of active agent delivery, in particular by way of using an electroactive polymer membrane to facilitate delivery of the desired agent. This disclosure further relates to iontophoresis and, more particularly, to the delivery of active agents such as therapeutic agents or drugs to a biological interface under the influence of electromotive force and/or current.

2. Description of the Related Art

Electroactive polymers (EAPs) are polymers whose shape, size or other characteristics are modified under application of a mechanical or electrical force. EAPs are useful as actuators or sensors, since they are typically capable of handling a large amount of force. One application of EAPs that is known in the art describes using EAPs as artificial muscles in robotics, such as those designed by NASA and described in U.S. Pat. Nos. 5,891,581 and 5,909,905, both of which are hereby incorporated by reference in their entireties.

Iontophoresis employs an electromotive force and/or current to transfer an active agent (e.g., a charged substance, an ionized compound, an ionic drug, a therapeutic, a bioactive-agent, and the like) to a biological interface (e.g., skin, mucus membrane, and the like), by using a small electrical charge applied to an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power station connected to the iontophoresis device via electrical leads. Each electrode assembly typically includes a respective electrode element to apply an electromotive force and/or current. Such electrode elements often comprise a sacrificial element or compound, for example silver or silver chloride.

The active agent may be either cationic or anionic, and the power source can be configured to apply the appropriate voltage polarity based on the polarity of the active agent. Iontophoresis may be advantageously used to enhance or control the delivery rate of the active agent. As discussed in U.S. Pat. No. 5,395,310 (hereby incorporated by reference in its entirety), the active agent may be stored in a reservoir such as a cavity. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their versatility and ease-of-use. An iontophoresis device that addresses one or more of these factors is desirable. Furthermore, a membrane or other regulator that may selectively alter the type and/or amount of active agent that is transported by active or passive means at a particular time and/or location is desirable.

The present disclosure is directed to overcoming one or more of the shortcomings set forth above, and providing further related advantages.

### BRIEF SUMMARY

The present disclosure relates to a controlled release membrane comprising an electroactive polymer. In at least one embodiment, the disclosure relates to a delivery device or vehicle comprising an electroactive polymer that utilizes passive or active transport to administer at least one active agent, such as a therapeutic or pharmaceutical agent or drug. In at least one embodiment, the disclosure relates to an iontophoresis device comprising an electroactive polymer wherein the device is operable to deliver at least one active agent to a biological interface such as skin or mucous membranes.

Passive transport may include but not be limited to, osmosis, diffusion, facilitated diffusion or other passive transport. One of skill in the art would recognize that passive transport occurs without any input of energy. Instead, passive transport is driven by kinetic energy possessed by the chemical composition itself. In certain aspects, passive transport entails the movement of molecules or ions across a membrane by moving toward a lower concentration and/or electrochemical gradient.

Active transport may include such things as membrane pumps (for example, a sodium/potassium pump), endocytosis and/or exocytosis, electroosmotic force, transport by iontophoresis, or transport by other means that requires energy output. In certain embodiments, carrier molecules or mediators may assist in passive or active transport to move ions and/or molecules across a subject's cell membranes. For example, iontophoresis employs an electromotive force to transfer an active agent such as an ionic drug or other therapeutic or active agent to a biological interface, such as skin or mucous membranes.

Iontophoresis devices typically comprise an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or comprise a sacrificial element or compound, for example silver or silver chloride.

The active agent, such as a therapeutic agent or pharmaceutical drug, may be a cation, an anion, or a mixture of both, and the power source can be configured to apply the appropriate voltage polarity based on the polarity of the active agent to be transported at a particular time and/or location. Iontophoresis may be advantageously used to enhance or control the delivery rate of the active agent. The active agent may be stored in a reservoir, such as a cavity. Alternatively, the active agent may be stored in a reservoir such as a porous structure or a gel. An ion exchange membrane (including a membrane comprising an electroactive polymer) may be positioned to serve as a polarity selective barrier between the active agent reservoir and the biological interface, thereby preventing backward flux of the oppositely charged ions from the skin or mucous membrane(s).

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a block diagram of an iontophoresis device comprising active and counter electrode assemblies according to one illustrated embodiment where the active electrode assembly comprises an ion exchange membrane (IEM) comprising an electroactive polymer, according to one illustrated embodiment.

FIG. 2 is a block diagram of the iontophoresis device of FIG. 1 positioned on a biological interface, with the outer release liner removed to expose the active agent according to one illustrated embodiment.

FIG. 3 is an isometric diagram of a transmucosal or transdermal delivery device that includes a patch 1 containing an active agent 2, an EAP membrane 3, and a medical backing 4, according to one illustrated embodiment.

FIG. 4 is a schematic diagram of the transdermal delivery device comprising an active electrode assembly and a counter electrode assembly and a plurality of microneedles according to one illustrated embodiment.

FIG. 5 is a bottom front isometric view of a plurality of microneedles in the form of an array according to one illustrated embodiment.

FIG. 6 is a bottom front isometric view of a plurality of microneedles in the form of one or more arrays according to another illustrated embodiment.

FIG. 7 is a flow diagram of a method for treating a subject including contacting the subject with a delivery device according to one illustrated embodiment.

FIG. 8 is a flow diagram of a method for making an active agent delivery device according to one illustrated embodiment.

### DETAILED DESCRIPTION

In the following description, certain specific details are set forth in order to provide a thorough understanding of various disclosed embodiments. One skilled in the relevant art, however, will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” or “in some embodiments” means that a particular referent feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an electrically powered device including “a power source” includes a single power source, or two or more power sources. It should also be noted that the term “or” is generally employed in its sense including “and/or” unless the content clearly dictates otherwise.

As used herein and in the claims, the term “membrane” means a layer, barrier, or material, which may, or may not be permeable. The term “membrane” may further refer to an interface. Unless specified otherwise, membranes may take the form a solid, liquid or gel, or any combination thereof and may or may not have a distinct lattice matrix or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. In addition, an ion selective membrane or charge selective membrane of the present disclosure may comprise an EAP. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS also from Tokuyama Co., Ltd. Further examples of ion exchange membranes are provided throughout the present disclosure.

As used herein and in the claims, the term “bipolar membrane” generally refers to a membrane that is selective to two different charges or polarities. Unless specified otherwise, a bipolar membrane may take the form of a unitary membrane structure a multiple membrane structure, or a laminate. The unitary membrane structure may have a first portion including cation ion exchange materials or groups and a second portion opposed to the first portion, including anion ion exchange materials or groups. The multiple membrane structure (e.g., two film) may be formed by a cation exchange membrane laminated, attached, or otherwise coupled to an anion exchange membrane. The cation and anion exchange membranes initially start as distinct structures, and may or may not retain their distinctiveness in the structure of the resulting bipolar membrane. Additionally, a bipolar membrane may comprise one or more EAPs.

As used herein and in the claims, the term “electroactive polymer” (EAP) generally refers to an electrically conductive polymer or a polymer that exhibits piezoelectric, pyroelectric and/or electrorestrictive properties in response to an electrical or mechanical force.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes (including electroactive polymer membranes), semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution. In at least one embodiment, two reservoirs may comprise solutions differing in electrolyte composition and separated by a membrane. In the even that the membrane comprises an EAP, the diffusion of ions between reservoirs can be reduced and the storage stability of the system increased by allowing the EAP to remain in a neutral state. However, when the EAP becomes charged, ions of the opposite charge can move readily across the membrane.

As used herein and in the claims, the term “active agent” refers to a compound, molecule, or treatment that elicits a biological response from any host, animal, vertebrate, or invertebrate, including, for example, fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., a cosmetic substance, and the like), a vaccine, an immunological agent, a local or general anesthetic or painkiller, an antigen or a protein or peptide such as insulin, a chemotherapy agent, and an anti-tumor agent.

In some embodiments, the term “active agent” refers to the active agent as well as to its pharmacologically active salts, pharmaceutically acceptable salts, prodrugs, metabolites, analogs, and the like. In some further embodiments, the active agent includes at least one ionic, cationic, ionizeable, and/or neutral therapeutic drug, and/or pharmaceutically acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. Alternatively, the active agent may be an “anionic active agent” which is negatively charged and/or is capable of forming negative charges in aqueous media. As a third possibility, the active agent may be of neutral charge and may convert to a positively or negatively charged agent once transport to or through the biological interface. In certain aspects, a neutral charged active agent may be assisted in transport by a carrier which may or may not be charged. Selection of the suitable active agents are well within the knowledge of one skilled in the art.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such drugs include lidocaine, articaine, and others of the caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes drugs such as exenatide; as well as peptides and proteins for treatment of obesity and other maladies.

Further non-limiting examples of active agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (NOVOCAINE®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “gel matrix” means a type of reservoir, which takes the form of a three-dimensional network, a colloidal suspension of a liquid in a solid, a semi-solid, a cross-linked gel, a non-cross-linked gel, a jelly-like state, and the like. In some embodiments, the gel matrix may result from a three-dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “subject” generally refers to any host, animal, vertebrate, or invertebrate, and includes fish, mammals, amphibians, reptiles, birds, and particularly, humans.

The headings provided herein are for convenience only and do not interpret the scope or meaning of the embodiments.

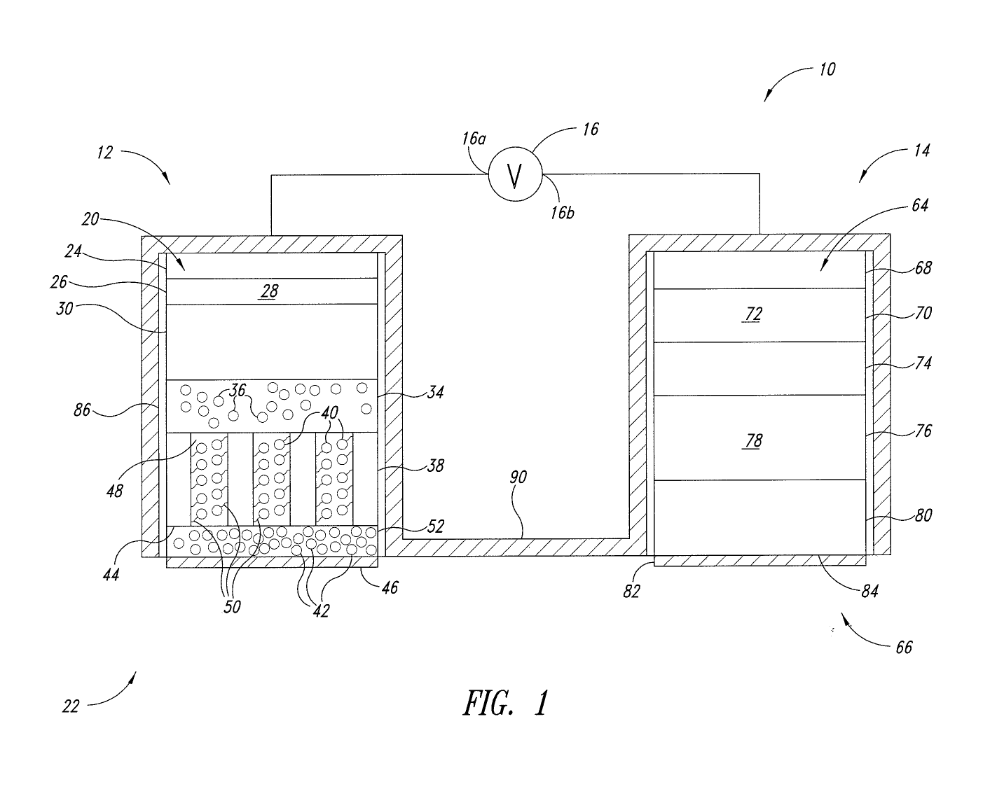

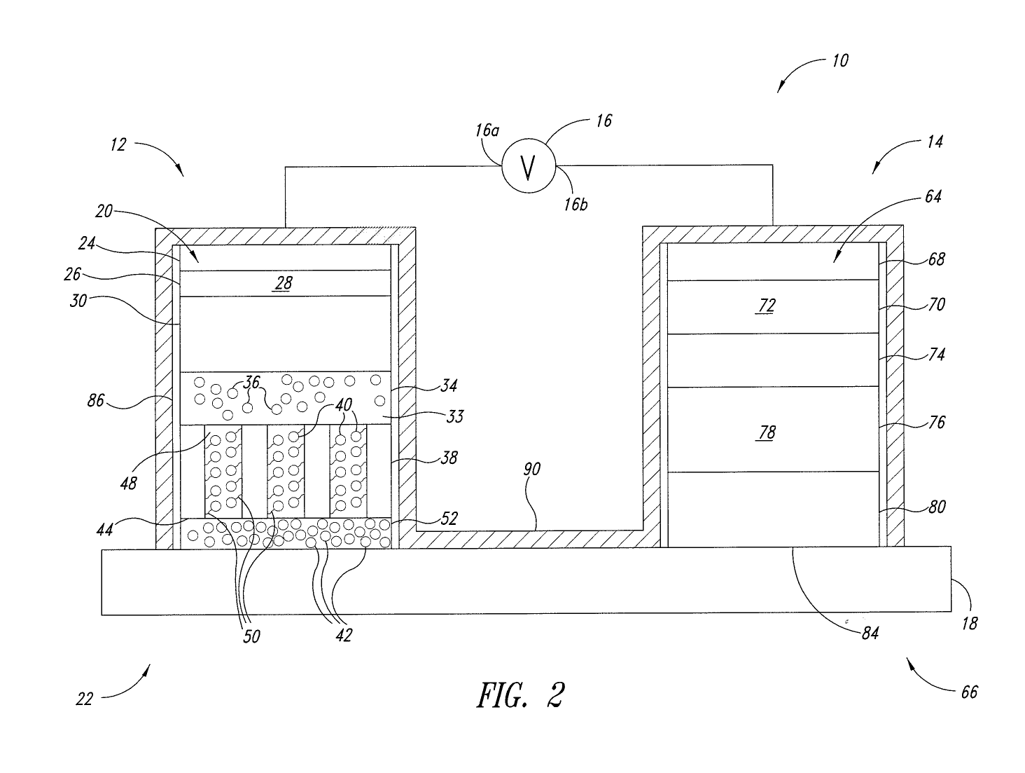

FIGS. 1 and 2 show a delivery device 10 in the form of an iontophoresis device comprising active and counter electrode assemblies 12,14, respectively, electrically coupled to a power source 16 to supply an active agent contained in the active electrode assembly 12 to a biological interface 18 (FIG. 2), such as a portion of skin or mucous membrane via iontophoresis, according to one illustrated embodiment.

In the illustrated embodiment, the active electrode assembly 12 comprises, from an interior 20 to an exterior 22 of the active electrode assembly 12: an active electrode element 24, an electrolyte reservoir 26 storing an electrolyte 28, an inner ion selective membrane 30 (which may optionally comprise an EAP), an inner active agent reservoir 34, storing active agent 36, an optional outermost ion selective membrane 38 (which may optionally comprise an EAP) that optionally caches additional active agent 40, an optional further active agent 42 carried by an outer surface 44 of the outermost ion selective membrane 38, and an optional outer release liner 46. The active electrode assembly 12 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological surface 18. Each of the above elements or structures will be discussed in detail below.

In some embodiments, the one or more active agent reservoirs 34 are loadable with a vehicle and/or pharmaceutical composition for transporting, delivering, encapsulating, and/or carrying the one or more active agents 36, 40, 42. In some embodiments, the pharmaceutical composition includes a therapeutically effective one or more active agents 36, 40, 42.

The active electrode element 24 is electrically coupleable via a first pole 16a to the power source 16 and positioned in the active electrode assembly 12 to apply an electromotive force to transport the active agent 36, 40, 42 via various other components of the active electrode assembly 12.

The active electrode element 24 may take a variety of forms. In one embodiment, the device may advantageously take the form of a carbon-based active electrode element 24. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and, in some embodiments, may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate. In some embodiments, the one or more electrolyte reservoirs 124 include an electrolyte 128 comprising at least one biologically compatible anti-oxidant selected from ascorbate, fumarate, lactate, and malate, or salts thereof.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to ascorbic acid (vitamin C), tocopherol (vitamin E), or sodium citrate.

As noted above, the electrolyte 28 may be in the form of an aqueous solution housed within a reservoir 26, or may take the form of a dispersion in a hydrogel or hydrophilic polymer capable of retaining substantial amount of water, along with an optional carrier. For instance, a suitable electrolyte may take the form of a solution of 0.5 M disodium fumarate: 0.5 M Poly acrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane and may optionally comprise an EAP. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the iontophoresis device 10. In certain aspects, when an EAP is used such ionic transfer prevention may be conducted by inducing a charge on the EAP or cycling between neutral state and charged state.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 1 and 2, take the form of an ion exchange membrane having pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). As indicated herein, any membrane utilized in one or more embodiments may comprise an EAP.

Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. In certain aspects, the outermost ion exchange membrane 38 may comprise an EAP. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38. In at least one embodiment, the outermost ion selective membrane 38 comprises one or more EAPs that may allow for selective transport or “pumping” of the active agent to the biological interface if an electrical and/or mechanical force is applied.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38 (which may optionally comprise an EAP). In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

In certain embodiments where one or more membranes may comprise one or more EAPs, the membranes may all contain the same charge or different charges, or different degrees of the same or different charges, thereby further able to regulate the transport of the active agent(s).

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2.

An interface coupling medium (not shown) may be employed between the electrode assembly and the biological interface 18. The interface coupling medium may, for example, take the form of an adhesive and/or gel. The gel may, for example, take the form of a hydrating gel. Selection of suitable bioadhesive gels are within the knowledge of one skilled in the art.

In the embodiment illustrated in FIGS. 1 and 2, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupleable via a second pole 16b to the power source 16, the second pole 16b having an opposite polarity to the first pole 16a. In one embodiment, the counter electrode element 68 is an inert electrode. For example, the counter electrode element 68 may be the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid. In certain aspects, wherein the electrolyte reservoir is bound on at least one side by a membrane, such membrane may comprise one or more EAPs.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78 and optionally comprises an EAP. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane and may optionally comprise an EAP. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore usually an anion exchange membrane, which substantially passes anions and blocks cations, thereby prevents the back flux of the cations from the biological interface. Examples of suitable ion exchange membranes are discussed above. For instance, a neutral membrane may be converted into an ion-exchange membrane, or the ion exchange membrane may be made from components that, upon polymerization, already contain the desired ion-exchange characteristics. Additional examples include membranes or other media comprising electroactive polymers (EAPs).

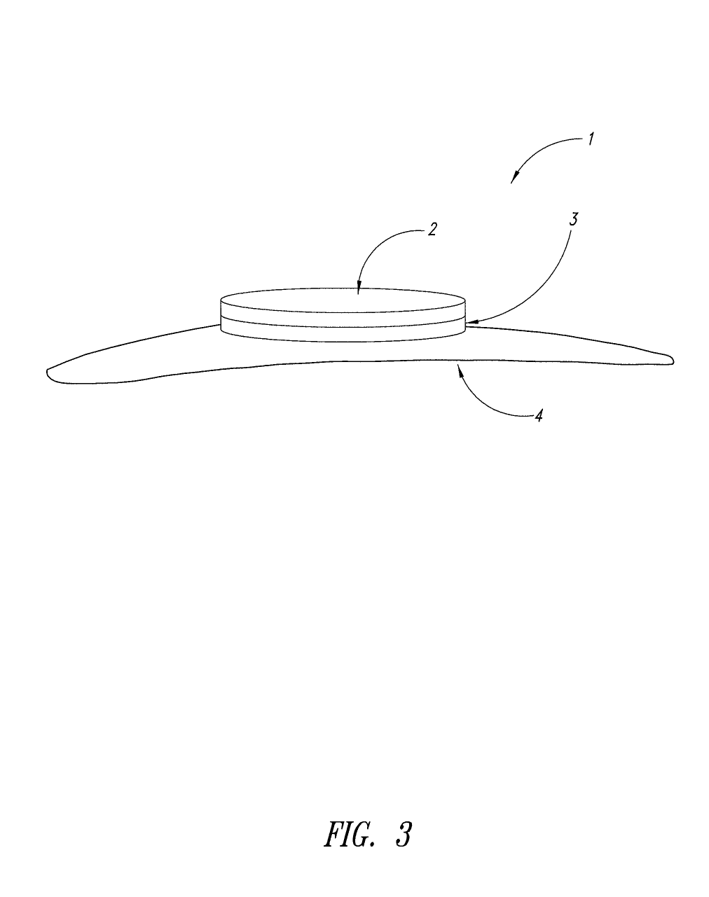

As shown in FIG. 3, in some embodiments, a transmucosal or transdermal delivery device may includes a patch 1 containing an active agent 2, an EAP membrane 3, and a medical backing 4.

Electroactive Polymers

Polymers are generally lightweight, tough, have high impact resistance, can be readily manufactured into large areas and may be cut and formed into complex shapes. Polymers also usually have a low dielectric constant, low elastic stiffness and low density, which result in a high voltage sensitivity and low acoustic and mechanical impedance. Polymers also generally possess a high dielectric breakdown and high operating field strength, which means that they can withstand much higher driving fields than ceramics. Polymers also offer the ability to pattern electrodes on a film surface, and or comprise selected regions of different polarity.

In general, most polymers are good insulators. However, there are certain polymers, called electroactive polymers (EAPs) that exhibit piezoelectric (such as amorphous, aromatic polyimides), pyroelectric or electrostrictive properties in response to electrical or mechanical forces. In general, EAPs can be deformed repetitively by applying an external electrical, mechanical or electro-mechanical force. Under certain circumstances, EAPs can be made highly conductive in the presence of certain additives, or dopants.

EAPs comprise several groups of materials including: a) conductive plastics (made from traditional thermoplastics containing fillers that render them conductive); b) inherently conductive polymers (which conduct electricity on their own after being “doped”); c) inherently dissipative polymers (which have been modified to become conductive; and d) other polymers with low dielectric constants that have potential in microelectronic applications.

EAPs can have several different configurations, but are generally grouped into two classes: dielectric EAPs and ionic EAPs. Dielectric EAPs, which typically require a large actuation voltage (on the magnitude of several thousand volts) but consume very little electrical power and require no or little power to keep the actuator at a given position. Another type of EAP is the ionic EAPs, in which actuation is caused by the displacement of ions inside the polymer. Only a few volts are typically needed for actuation (generally about 1-3.5 V), but the ionic flow implies a higher electrical power needed for actuation and energy is usually needed to keep the actuator at a given position. Any materials that are conductive, electroactive or can be made to be conductive or electroactive may be used for making EAPs.

Some examples of EAPs include but are not limited to poly(sulfur nitride); polyacetylene (which can be reduced or oxidized to have electronic properties of metals); poly(ethylenedioxythiophene); poly(p-phenylene); poly(p-phenylenevinylene); poly-1,6 heptadiyne; polyphenylene sulfide; poly-m-phenylene; polyaniline; polypyrrole; polythiophene; polyisoprene; polyfuran; polyimides; polythiophenes; ionomeric polymer metal composites, carbon nanotubes, ferroelectric polymers, ionic polymer gels, vinyl copolymers; odd-numbered nylons; poly(vinylidene fluoride) (PVDF), as well as its copolymers with trifluoroethylene and tetrafluoroethylene which contain a crystalline component in an inactive amorphous phase; polythioureas; nitrile substituted polymers, such as polyacrylonitrile (PAN), poly(vinylidenecyanide vinylacetate) (PVDCNNAc), polyphenylethernitrile (PPEN), poly(1-copolymers); polyvinylchloride (PVC); a graft copolymer with a fluorine-containing backbone and a carbazole-containing side chain, such as that described in U.S. Patent Publication No. 20040127986 (hereby incorporated by reference in its entirety); perfluoride compounds, such as Nafion®, described in U.S. Pat. No. 6,109,852 (hereby incorporated by reference in its entirety); any combination of these, and the like. Some of these listed examples of electroactive polymers are described in U.S. Pat. No. 4,519,938 and U.S. Patent Publication No. 20030166773, both of which are hereby incorporated by reference in their entireties.

An EAP membrane of any thickness, any density, any porosity, any size, any shape and any form may be used with certain aspects of the present disclosure. In addition, any number of EAP membranes may be utilized, as necessary or as desired in the delivery devices described herein. Thin films, such as those made from polypyrrole and polyaniline, demonstrate exceptional combinations of mechanical strength, actuation stress and actuation strain, as well as minimize the time required for ions to diffuse into or out of the membrane. For example, polypyrrole performance has been measured at 30-50 Mpa strength, 4 MPa actuation stress and 2.4% strain for long-life in-plane linear actuation of a thin (approximately 10 micrometers) film immersed in liquid electrolyte. (Malone, et al., Solid Freeform Fab. Proc., 2004, hereby incorporated by reference in its entirety).

Without wishing to be bound by any particular theory, some electroactive polymers may be generated by modifying the conductivity of a polymer with one or more electron acceptor and/or electron donor doping reactions. In one example, polypyrrole may be prepared by electrolytic oxidation of pyrrole, inducing a charge along the polymer backbone of the molecule. In one such example, a net negative charge is induced along the polymer backbone, which results in a film capable of behaving as a cationic ion exchange membrane. In other examples, a net positive charge is induced along the polymer backbone, which results in a film capable of behaving as an anionic ion exchange membrane. Thus, the present disclosure envisions use of an ion exchange membrane wherein the membrane comprises a positively or negatively charged electroactive polymer.

In addition, the amount of charge induced along the polymer backbone may vary according to the particular method of synthesis. For example, the positive or negative charge along the polymer backbone of EAPs generated by oxidation or reduction reactions will vary according to the amount of reaction allowed to occur. Varying the charge along the polymer backbone may impact the structure and/or function of the EAP membrane. It is also possible to synthesize EAPs by electropolymerization of aromatic heterocyclic compounds.

Specific EAPs can be formed from a conductive polymer doped with surfactant molecules (such as sodium dodecyl benzene sulfonate) or from an ionic polymer metal composite (which typically comprise perfluorosulfonate polymers that contain sulfonic or carboxylic ionic functional groups. One example of such a polymer is Nafion® which is a poly(tetrafluoroethylene) based ionomer produced in a sheet geometry with positive counter ion (for example, sodium or lithium) contained in the matrix. Typically, the outer surface region (usually less than a micrometer) of the polymer sheet is impregnated with a conductive metal such as platinum or gold. The resulting EAP polymer is capable of absorbing water until its physical ability to expand is balanced by the affinity of water for the polymer-fixed ions and free counter ions. When a mechanical or electrical force is applied across the EAP, movement of water and mobile ions (in the case of an electrical field) deforms the EAP. When the force ceases, the EAP returns to approximately its original size or shape.

Dopants are well known in the art and may be, for example, electron acceptors (such as arsenic pentafluoride), or halogen or electron donors (such as alkali metals), although acids have also been used (such as hydrochloric acid). Conductivity typically varies with the type and dopant concentration used, method of synthesis, as well as general morphology of a compound (configurational as well as conformational factors, including crystallinity). For example, the conductivity of polyacetylene film is increased significantly by stretching in the direction of molecular alignment.

Some electroactive polymers may also be generated as continuous films from solutions of monomer by anodic polymerization in an electrochemical cell, with the dopant ions introduced directly from the monomer solution. In at least one embodiment, the EAP has a molecular weight sufficient that films of the material will maintain mechanical integrity in an electrolyte solution. The molecular weight required will vary according to the structure of the polymer and the solvent used. In at least one embodiment, the molecular weight of the polymer is in the range of about 5,000-10,000 Daltons or greater.

In addition to the many modes of synthesizing EAPs, neutral but potentially ionomeric materials may be grafted onto neutral membrane polymers even as a post membrane-formation act, to form an ion-exchange membrane (including electrostrictive-grafted elastomers comprising a flexible backbone combined with a grafted polymer that can be produced in a crystalline form). For example, polyacrylate ester can be grafted onto cellophane and subsequently hydrolyzed to form a weak-acid cationic exchange membrane. In another example, polystyrene may be grafted onto polyethylene and sulfonated, to form a strong-acid cationic exchange membrane. One method of post membrane formation grafting reactions described in U.S. Pat. No. 5,238,613 (hereby incorporated by reference in its entirety), creates free radicals on the pore surfaces which act as initiation sites for polymerization of added monomers (that are able to easily diffuse to the initiation sites). The free radicals can be generated, for example, by grafting sites by peroxides or redox catalysts, or by exposure to electrons, gamma rays or UV radiation.

Various types of electroactive materials included in the disclosure, such as polyimide, have the capacity of accepting electrons from another material or chemical entity at a finite rate without itself undergoing a change which limits this capacity. The particular chemical chosen may have molecular, ionic, atomic or adjacent redox sites within or in contact with the polymer. The redox potential of the polymer is positive to the reduction potential of the chemical entity, thereby permitting the polymer to readily accept the electrons. The polymer must possess chemical functionality (such as redox sites) whose redox potential is positive relative to the redox potential of the chemical entity. Examples of such functional groups include the aromatic imide groups of modified or unmodified polyimides (some examples include polyethylene terephthalates, polyamide-imide-esters, polyamide-imides, polysiloxane-imides, fluorocarbon-containing polyimides, as well as other mixed polyimides or polyimide blend materials), whose reduction potentials are more positive than the oxidation potential of the reduced electroactive polymer. Other electroactive polymers with aromatic carbonyl moieties include terephthalate-containing polymers such as Mylar®. This electroactive polymer functionality must be reversibly redox active so that it is capable of accepting and donating electrons rapidly and without competing, irreversible chemical changes. The reversibility may require such precautions as exclusion of oxygen, potential proton donors or nucleophilic and electrophilic reagents. The electroactive polymer must also be able to take up sufficient solvent by swelling or absorption to permit diffusion of electrolyte ions into the polymer.

In addition, compounds suitable for redox reactions are well known in the art and can be generated electrochemically by one of skill in the art.

Electroactive polymers may be utilized in transdermal or transmucosal drug delivery vehicles that rely on passive diffusion to deliver at least one active agent, or vehicles that utilize iontophoretic means of drug delivery, both of which are described in the present disclosure. EAPs may be present in the drug delivery device in any form, including but not limited to a membrane, semisolid, colloid, matrix, hydrogel, polymer gel, dispersion, solution, thin film, a liquid electrolyte surrounded by some kind of encapsulation, any combination of these, or any other form.

If the EAP is used with a passive transport delivery vehicle, the EAP may be deformed upon electrolyte imbalance with the subject to which it is adhered. Alternatively, if the EAP is used with an active transport delivery vehicle, the EAP can be deformed upon application of a voltage (generally about 1-3.5 V for ionic EAPs).

Typically, EAP deflection varies linearly at low voltages (usually about <1 V) and nonlinearly at higher voltages. An EAP can deform at a rate of about 20-35 degrees/volt, with the magnitude of deflection of the EAP typically being similar in both directions, upon reversing the polarity of the electric field. This suggests that the EAP surfaces have similar conductivity and that the EAP composition is reasonably uniform. However, it is possible for EAPs to deflect significantly more in one direction, particularly if ions leach out or the EAP has imperfections in it. Improved surface conductivity may be accomplished by using metal vapor deposition or other doping methods for generating EAPs. In addition, EAPs may contain an optionally impermeable surface, since EAPs typically behave differently in air than in water, other liquids or electrolyte solutions.

In at least one embodiment, regardless of the type of pharmaceutical drug delivery vehicle used with the EAP, the EAP membrane may be temporarily deformed by applying a mechanical and/or electrical force to the membrane, particularly a cyclical force, thereby allowing for cyclical transport of at least one active agent. In one example, the EAP membrane is cycled between neutral and a charged state by applying a mechanical and/or electrical force, thereby allowing for cyclical transport of at least one active agent. In at least one embodiment, the active agent is charged, thus allowing for administration when the EAP is oppositely charged. For example, if an EAP membrane is positively charged and at least one active agent is negatively charged, cycling the EAP from neutral to positively charged would create a “pumping” mechanism that transports the agent to the subject. As indicated herein, the charge on the EAP can be varied as desired, thus varying the strength of the “pumping” action. Such EAP membranes may be used in addition to or instead of other membranes, including other ion exchange membranes. In addition, cycling the EAP membrane from a neutral to charged state can control any passive diffusion or “leaking” of active agent within or between reservoirs, for example in an iontophoretic device as described herein. In this manner, the EAP membrane may also provide a selective barrier between reservoirs and/or between an active agent and contact with the subject. The EAP may also be cycled between charges, for example between positively and negatively charged, or between negatively charged and positively charged, depending on the charge of the EAP as well as the charge of the active agent desired to be transported or administered.

Furthermore, in any aspect wherein an electrical field is applied to an EAP, the electrical field (current or voltage) may be constant or fixed, variable, cyclical, any combination thereof, or otherwise.

In certain aspects, the active agent and/or carrier may be physically or chemically associated with the EAP membrane (such as by covalent bonding, noncovalent bonding, ionic bonding, magnetic bonding, cross-linking, Van der Waals forces, entrapment, electrostatic linking, etc.), and may thereby be released upon applying an electrical or mechanical force. As indicated, any membrane of the present disclosure may comprise one or more EAPs, including the outermost ion selective membrane 80.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The iontophoresis device 10 may further comprise an inert molding material 86 adjacent exposed sides of the various other structures forming the active and counter electrode assemblies 12,14. The molding material 86 may advantageously provide environmental protection to the various structures of the active and counter electrode assemblies 12,14. Enveloping the active and counter electrode assemblies 12,14 is a housing material 90.

As best seen in FIG. 2, the active and counter electrode assemblies 12,14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may, for example, take the form of an adhesive and/or gel. The gel may, for example, take the form of a hydrating gel or a hydrogel. If used, the interface-coupling medium should be permeable by the active agent 36, 40, 42.

The power source 16 may take the form of one or more chemical battery cells, super- or ultra-capacitors, fuel cells, and the like. In some embodiments, the power source 16 takes the form of at least one primary cell or secondary cell. Other suitable examples of power source 14 include at least one of a button cell, a coin cell, an alkaline cell, a lithium cell, a lithium ion cell, a zinc air cell, a nickel metal hydride cell, and the like. In some embodiments, the power source 16 takes the form of at least one printed battery, energy cell laminate, thin-film battery, power paper, and the like, or combinations thereof.

In some embodiments, the source 16 is selected to provide sufficient voltage, current, and/or duration to ensure delivery of the one or more active agents 36, 40, 42 from the reservoir 34 and across a biological interface (e.g., a membrane) to impart the desired physiological effect.

The power source 16 may, for example, provide a voltage of 12.8 V DC, with tolerance of 0.8 V DC, and a current of 0.3 mA. The power source 16 may be selectively electrically coupled to the active and counter electrode assemblies 12, 14 via a control circuit, for example, via carbon fiber ribbons. The iontophoresis device 10a may include discrete and/or integrated circuit elements to control the voltage, current and/or power delivered to the electrode assemblies 12, 14. For example, the iontophoresis device 10 may include a diode to provide a constant current to the electrode elements 24, 68.

As suggested above, the active agent 36, 40, 42 may take the form of one or more ionic, cationic, anionic, ionizeable, and/or neutral drug or other therapeutic agent. In certain aspects, the active agent is neutral in charge and may be assisted in transport by a carrier that is physically and/or chemically associated with the active agent and which may or may not be charged. Consequently, the poles or terminals of the power source 16 and the selectivity of the outermost ion selective membranes 38, 80 and inner ion selective membranes 30, 74 are selected accordingly.

Carriers

Carriers, and particularly pharmaceutical carriers, as described by the present disclosure, may be used with either a passive or active transport delivery device and may include a degradable or nondegradable polymer, hydrogel, organogel, liposomes, micelles, microspheres, cream, lotion, paste, gel, ointment or other matrix that allows for transport of an agent across the skin or mucous membranes of a subject. In certain aspects, the carrier is positively or negatively charged. In at least one embodiment, the carrier allows for controlled release formulations of the compositions disclosed herein.

As one of skill in the art would appreciate, the pharmaceutical formulations will be readily understood in the art. For example, ointments may be semisolid preparations based on petrolatum or other petroleum derivatives. Emulsions may be water-in-oil or oil-in-water and include, for example, cetyl alcohol, gylceryl monostearate, lanolin and steric acid, and may also contain polyethylene glycols. Creams may be viscous liquids or semisolid emulsions of oil-in-water or water-in-oil. Gels may be semisolid suspensions of molecules including organic macromolecules as well as an aqueous, alcohol and/or oil phase. Some such organic macromolecules include but are not limited to, gelling agents, (such as carboxypolyalkylenes), hydrophilic polymers, such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose, phthalate, and methyl cellulose, tragacanth or xanthan gums, sodium alginate, gelatin, and others, or any combination thereof.

In certain embodiments, the carrier may be a liposome, micelle, or microsphere, or other small lipid-based carrier. One of skill in the art would readily understand such formulations, and appreciate that they may be used by incorporation into the reservoir of the delivery device, or formulated to be applied directly to a subject's body surface or other interface. For example, liposomes may be microscopic vesicles having a lipid wall comprising a lipid bilayer, and may be preferred for poorly soluble or insoluble therapeutic agents. Liposomal formulations may be cationic, anionic, or neutral preparations. Materials and methods of making such liposomal preparations are well known in the art.

In certain embodiments, micelles may be used as the carrier. As one of skill in the art would appreciate, micelles are comprised of surfactant molecules arranged with polar headgroups forming an outer shell, while the hydrophobic hydrocarbon chains are oriented toward the middle of the sphere, forming a core. Micelles may form from surfactants such as potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol-10 and nonoxynol-30, among others.

In certain embodiments, the carrier is an organogel. In at least one embodiment, the carrier is a lecithin organogel. Lecithin (1,2-diacyl-sn-3-phosphocholine) is derived from soybeans or eggs, and forms an organogel that is clear, thermodynamically stable, non-polymeric, viscoelastic, isotropic and biocompatible gel when mixed with organic liquid (such as isopropyl palmitate, n-decane or isopropyl myristate) and a polar solvent (such as water). Lecithin organogels are rich in phospholipids that are entangled reverse cylindrical micelles. Various forms of lecithin organogels are described in Kumar and Katare (“Lecithin Organogels as a Potential Phospholipid-Structured System for Topical Drug Delivery: A Review”, AAPS PharmSciTech 2005; 6(2) Article 40), which is hereby incorporated by reference in its entirety.

Briefly, the organogel matrix chiefly consists of a surfactant (lecithin) as gelator molecules, a nonpolar organic solvent as external or continuous phase, and a polar agent, usually water. Many varieties of organic solvents are able to form a gel in the presence of lecithin. Included are linear, branched, and cyclic alkanes; ethers and esters; fatty acids; and amines. Some specific examples include ethyl laureate, ethyl myristate, isopropyl myristate, isopropyl palmitate, cyclopentane, cyclooctane, trans-decalin, trans-pinane, n-pentane, n-hexane, n-hexadecane, and tripropylamine, as well as others.

The polar agent of the organogel acts as a structure forming and stabilizing agent. Water is the most commonly employed polar agent, although some other polar solvents such as glycerol, ethylene glycol, and formamide also may be used.

In addition, at least one embodiment also includes incorporation of synthetic polymer (i.e., pluronics) in lecithin organogels. Pluronics are also known as poloxamers, poloxamer polyols or lutrols, and refer to a series of nonionic, closely related block copolymers, such as of ethylene oxide and/or propylene oxide. Pluronics may be useful as cosurfactants, emulsifiers, solubilizers, suspending agents and/or stabilizers.

Other Components

Any embodiment may further comprise one or more additional ingredients, such as one or more thickening agents, medicinal agents, growth factors, wound-healing factors, peptidomimetics, proteins or peptides, carbohydrates, bioadhesive polymers, preservatives, inert carriers, lipid absorbents, chelating agents, buffers, anti-fading agents, stabilizers, moisture absorbents, vitamins, caffeine or other stimulants (such as epinephrine, adrenaline, norepinephrine, etc.), UV blockers, humectants, cleansers, colloidal meals, abrasives, herbal extracts, phytochemicals, fragrances, colorants or dyes, film-forming materials, analgesics, etc. A single excipient may perform multiple functions or a single function. One of skill in the art will readily be able to identify and choose any such excipients based on the desired physical and chemical properties of the final formulation. Furthermore, temperature changes may be employed as part of the administration of the carrier. For example, the carrier may be heated or cooled to vary the dosage delivered or taken up by the subject's body surface.

Examples of some commonly used thickening agents include, but are not limited to, cellulose, hydroxypropyl cellulose, methyl cellulose, polyethylene glycol, sodium carboxymethyl cellulose, polyethylene oxide, xanthan gum, guar gum, agar, carrageenan gum, gelatin, karaya, pectin, locust-bean gum, aliginic acid, bentonite carbomer, povidone and tragacanth, or any combination thereof.

One of skill in the art would also readily be able to identify and choose any optional medicinal agents or their pharmaceutically acceptable salts, based on the desired effect for the final formulation. Examples of medicinal agents include, but are not limited to, antifungal compositions (ciclopirox, triacetin, nystatin, tolnaftate, miconizole, clortrimazole, and the like), antibiotics (gentamicin, polymyxin, bacitracin, erythromycin, and the like), antiseptics (iodine, povidine, benzoic acid, benzyol peroxide, hydrogen peroxide, and the like), and anti-inflammatory compositions (hydrocortisone, prednisone, dexamethasone, and the like), or any combination thereof.

One of skill in the art would also readily identify and choose any optional bioadhesive polymers that may be useful for hydrating the skin and increasing pharmaceutical delivery. Some examples of bioadhesive polymers include, but are not limited to, pectin, alginic acid, chitosan, hyaluronic acid, polysorbates, polyethylene glycol, oligosaccharides, polysaccharides, cellulose esters, cellulose ethers, modified cellulose polymers, polyether polymers and oligomers, polyether compounds (block copolymers of ethylene oxide and propylene oxide) polyacrylamide, poly vinyl pyrrolidone, polymethacrylic acid, polyacrylic acid, or any combination thereof.

One of skill in the art would readily appreciate any number of analgesics may be employed. For example, ketoprofen, piroxicam, ibuprofen, lidocaine, novocaine, morphine, codeine, and the like, may be used individually or in combination.

One of skill in the art would also recognize that the teachings herein may be utilized with wounded or intact skin, or on mucous membranes, including but not limited to oral, bronchial, vaginal, rectal, uterine, urethral, otic, ophthalmologic, pleural, nasal, or the like.

Methods of Making a Passive Transport Delivery Device

One of skill in the art would readily understand that any number of methods of making the disclosed embodiments may be employed.

For example, the compositions, including pharmaceutical compositions may be prepared according to standard protocols, which are well known in the art. For example, the methods recited in 1 Remington: “The Science and Practice of Pharmacy” 289 (Alfonso R. Gennaro ed., 19th ed. 1995), hereby expressly incorporated by reference in its entirety, can be used.

Another example includes separating the solutions into water soluble and oil soluble components. The water soluble components can be mixed together in one container while the oil soluble components can be mixed together in a separate container, and each mixture heated individually to form a solution. The two solutions may then be mixed and the mixture allowed to cool. Such compositions may be packaged in, for example, a patch or bandage and stored, or used directly. Other exemplary embodiments are set forth in the Examples section herein.

Such delivery device may comprise a reservoir, an absorbent layer, a medical backing, an adhesive, one or more membranes (including one or more membranes comprising an EAP), or other structures. The medical backing and adhesive may be located on various positions of the delivery device. For example, the medical backing and adhesive may be adjacent to the carrier and/or therapeutic agent, may be opposing the carrier and/or therapeutic agent, or may be intermixed with the carrier and/or therapeutic agent.

In at least one embodiment, the device can take the form of a patch of any size or shape. Suitable patches include, but are not limited to, the matrix type patch, the reservoir type patch, the multi-laminate drug-in-adhesive type patch, and the monolithic drug-in-adhesive type patch. These and others are readily known in the art and are further described in Tapash, et al, (Transdermal and Topical Drug Delivery Systems, 1997), hereby incorporated by reference in its entirety.

For example, a matrix patch may comprise a therapeutic agent containing matrix, an adhesive backing film overlay, and a release liner. In some embodiments, one or more impermeable or semipermeable layers or membranes may be used to minimize drug migration into the backing. The matrix may be held against a subject's body surface by the adhesive overlay. Examples of suitable matrix materials include, but are not limited to, lipophilic polymers, hydrophilic polymers, hydrogels, or polyvinylpyrrolidone/polyethylene oxide mixtures.

In certain embodiments, the reservoir type patch may comprise a backing film coated with an adhesive, and a reservoir compartment comprising a therapeutic agent formulation which may or may not be separated from the subject's body surface by one or more semipermeable membranes.

In certain embodiments, the monolithic drug-in-adhesive patch may comprise a drug formulation in the skin contacting adhesive layer, a backing film, and possibly a release liner. The adhesive may function to release the anesthetic and/or adhere the anesthetic matrix to the skin. The drug-in-adhesive system does not require an adhesive overlay and thus the size and height of the patch may be minimized.

In certain embodiments, the multi-laminate drug-in-adhesive patch may further incorporate one or more semipermeable membrane between two distinct drug-in-adhesive layers or multiple drug-in-adhesive layers under a single backing film.

In certain embodiments, the delivery device further comprises an adhesive. Adhesives are well known in the art and include, but are not limited to, polyisobutylene-based adhesives, silicone-based adhesives, and acrylic-based adhesives. The adhesive may be based on natural or synthetic rubber. In certain embodiments, the device further comprises a pressure sensitive adhesive. Pressure sensitive adhesives generally adhere to a substrate by applying light force, and usually do not leave a residue when removed.

In certain embodiments, the device may be prepared by casting a fluid admixture of adhesive, therapeutic agent and carrier onto a backing layer, followed by lamination of the release liner. In certain embodiments, the adhesive mixture may be cast onto the release liner, followed by lamination of the backing layer. In certain embodiments, the drug reservoir may be prepared in the absence of therapeutic agent and then loaded by saturating or soaking it in the therapeutic agent and/or a carrier. Other methods of making include solvent evaporation, film casting, melt extrusion, thin film lamination, die cutting, or the like.

In certain embodiments, the medical backing layer may function as the primary structural element of the delivery device and may provide the device with flexibility and occlusivity (which allows the subject's body surface to become hydrated with use of the device), or permeability (which allows the subject's body surface to encounter other atmospheric agents). The backing may comprise a flexible elastomeric material that protects and/or prevents the composition contained in the device.

In certain embodiments, the medical backing and/or adhesive extends beyond the surface of the device reservoir, which allows for adherence to the subject's body even once the treatment site has become hydrated.

One of skill in the art would recognize that the carrier (including an organogel) and/or the immunity agent may be contained within a delivery device, such as a patch, bandage, reservoir, rupturable membrane, application chamber, tape, film, or other delivery device that allows for transdermal or transmucosal delivery of the agent. In at least one embodiment, the carrier and/or the immunity agent is contained within a patch. In at least one embodiment, the carrier and/or the immunity agent is impregnated on a substrate contained within a patch. In at least one embodiment, a substrate contained within the patch is saturated with the carrier and/or the immunity agent. In at least one embodiment, a substrate contained within a patch is an absorbent layer saturated with the carrier and/or the immunity agent. In at least one embodiment, the carrier and/or immunity agent is contained on a bandage, patch, film or the like, and may comprise or be joined with an adhesive. In at least one embodiment, the patch or other delivery device further comprises an adhesive backing that allows the device to adhere to a subject's body.

One of skill in the art would recognize that multiple materials may be used for an absorbent layer within a patch, including fabric, fibers, particulate matter, or other solid support that is capable of absorbing a carrier and/or an immunity agent. Some examples of materials used in constructing the absorbent layer may include, but not be limited to, cotton, polyester, polyfil, other natural or synthetic materials or any combination thereof.

One of skill in the art would recognize that other such microstructures may be employed. In certain aspects, abrasive agents may be utilized in order to increase the transdermal or transmucosal delivery of the therapeutic agent. One of skill in the art would recognize that a variety of abrasive means may be employed, such as physical, chemical, radiation, mechanical, structural or other such means. Examples of abrasive agents that may be employed include but are not limited to temperature changes; such as heat or cold; light; magnets; chemical irritants such as acids, bases, alcohols or other solvents, polymers (such as propylene glycol), salts (such as sodium laurel sulfate), plant compounds (such as from poison ivy or poison sumac), epoxy resins; vasoconstrictors such as epinephrine, adrenaline, norepinephrine; similar irritants or abrasives, and any combination thereof.

Additionally, abrasive agents may be utilized in order to increase the transdermal or transmucosal delivery of the therapeutic agent. One of skill in the art would recognize that a variety of abrasive means may be employed, such as physical, chemical, radiation, mechanical, structural or other such means. Examples of abrasive agents that may be employed include but are not limited to temperature changes; such as heat or cold; light; magnets; chemical irritants such as acids, bases, alcohols or other solvents, polymers (such as propylene glycol), salts (such as sodium laurel sulfate), plant compounds (such as from poison ivy or poison sumac), epoxy resins; vasoconstrictors such as epinephrine, adrenaline, norepinephrine; similar irritants or abrasives, and any combination thereof.

One of skill in the art may also appreciate that the transdermal or transmucosal delivery device may be more or less effective depending on the location on the subject. For example, highly vascularized areas may allow for greater delivery of the therapeutic agent, as would a surface that is wounded, for example by burn, laceration or abrasion. By contrast, areas that are not highly vascularized may allow for a slower or more gradual release of the therapeutic agent.

Agent Delivery by Iontophoresis

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular active agents may thus be carried through the biological interface into the underlying tissues primarily via electroosmotic solvent flow. In certain embodiments, the high molecular weight polyelectrolytic active agents may be proteins, polypeptides, or nucleic acids.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiment may include a control circuit or subsystem to control a voltage, current or power applied to the active and counter electrode elements 20, 68. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

Microstructures

The iontophoresis device discussed above may advantageously be combined with other microstructures, for example, microneedles.

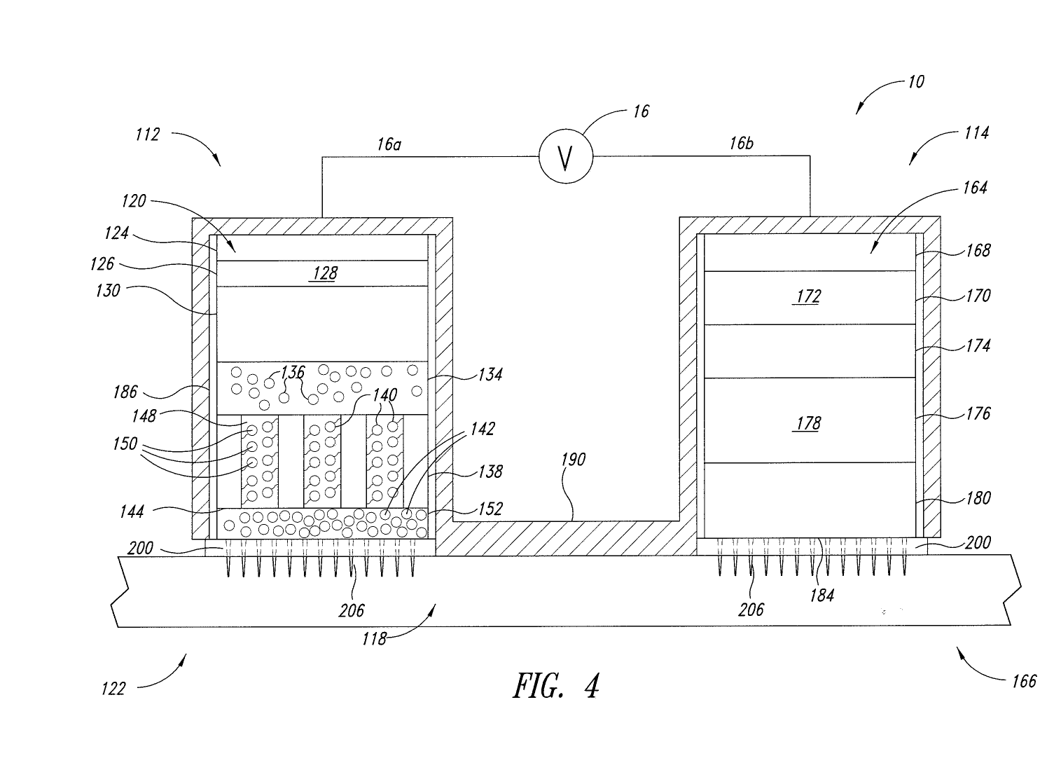

As shown in FIG. 4, the iontophoresis device 10 may further include a substrate 200 including a plurality of microneedles 206 in fluidic communication with the active electrode assembly 112, and positioned between the active electrode assembly 112 and the biological interface 118. The substrate 200 may be positioned between the active electrode assembly 112 and the biological interface 118. In some embodiments, the at least one active electrode element 120 is operable to provide an electromotive force to drive an active agent 136, 140, 142 from the at least one active agent reservoir 134, through the plurality of microneedles 206, and to the biological interface 118.

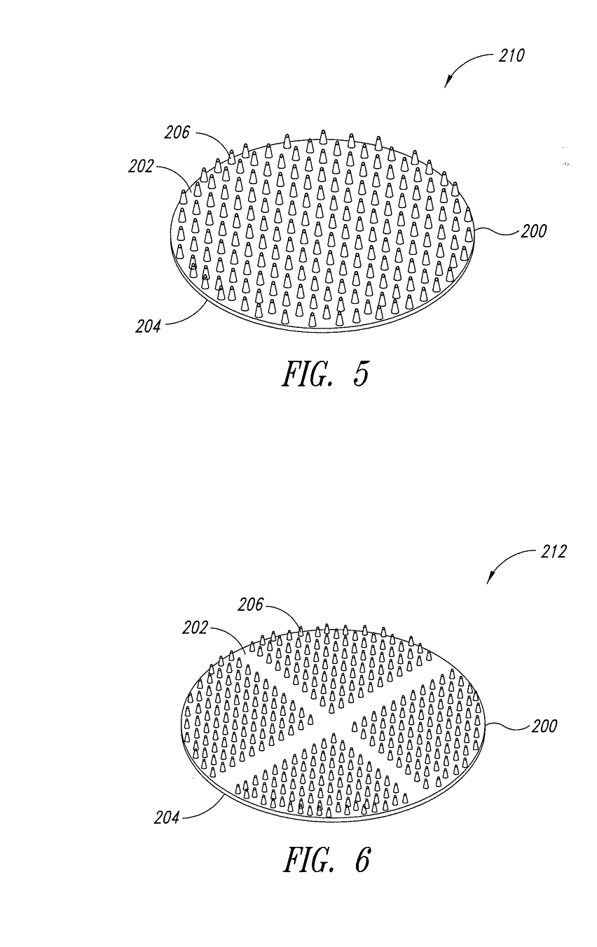

As shown in FIGS. 5 and 6, the substrate 200 includes a first side 202 and a second side 204 opposing the first side 202. The first side 202 of the substrate 200 includes a plurality of microneedles 206 projecting outwardly from the first side 202. The microneedles 206 may be individually provided or formed as part of one or more arrays. In some embodiments, the microneedles 206 are integrally formed from the substrate 200. The microneedles 206 may take a solid and permeable form, a solid and semi-permeable form, and/or a solid and non-permeable form. In some other embodiments, solid, non-permeable, microneedles may further comprise grooves along their outer surfaces for aiding the transdermal delivery of one or more active agents. In some other embodiments, the microneedles 206 may take the form of hollow microneedles. In some embodiments, the hollow microneedles may be filled with ion exchange material, ion selective materials, permeable materials, semi-permeable materials, solid materials, and the like.

The microneedles 206 are used, for example, to deliver a variety of pharmaceutical compositions, molecules, compounds, active agents, and the like to a living body via a biological interface, such as skin or mucous membrane. In certain embodiments, pharmaceutical compositions, molecules, compounds, active agents, and the like may be delivered into or through the biological interface. For example, in delivering pharmaceutical compositions, molecules, compounds, active agents, and the like via the skin, the length of the microneedle 206, either individually or in arrays 210, 212, and/or the depth of insertion may be used to control whether administration of pharmaceutical compositions, molecules, compounds, active agents, and the like is only into the epidermis, through the epidermis to the dermis, or subcutaneous. In certain embodiments, the microneedle 206 may be useful for delivering high-molecular weight active agents, such as those comprising proteins, peptides and/or nucleic acids, and corresponding compositions thereof. In certain embodiments, for example, wherein the fluid is an ionic solution, the microneedles 206 can provide electrical continuity between the portable power supply system 16 and the tips of the microneedles 206. In some embodiments, the microneedles 206, either individually or in arrays 210, 212, may be used to dispense, deliver, and/or sample fluids through hollow apertures, through the solid permeable or semi permeable materials, or via external grooves. The microneedles 206 may further be used to dispense, deliver, and/or sample pharmaceutical compositions, molecules, compounds, active agents, and the like by iontophoretic methods, as disclosed herein.

Accordingly, in certain embodiments, for example, a plurality of microneedles 206 in an array 210, 212 may advantageously be formed on an outermost biological interface-contacting surface of a delivery device 10. In some embodiments, the pharmaceutical compositions, molecules, compounds, active agents, and the like delivered or sampled by such delivery device 10 may comprise, for example, high-molecular weight active agents, such as proteins, peptides, and/or nucleic acids.

In some embodiments, a plurality of microneedles 206 may take the form of a microneedle array 210, 212. The microneedle array 210, 212 may be arranged in a variety of configurations and patterns including, for example, a rectangle, a square, a circle (as shown in FIG. 5), a triangle, a polygon, a regular or irregular shapes, and the like. The microneedles 206 and the microneedle arrays 210, 212 may be manufactured from a variety of materials, including ceramics, elastomers, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon rubbers, silicon-based organic polymers, superconducting materials (e.g., superconductor wafers), and the like, as well as combinations, composites, and/or alloys thereof. Techniques for fabricating the microneedles 206 are well known in the art and include, for example, electro-deposition, electro-deposition onto laser-drilled polymer molds, laser cutting and electro-polishing, laser micromachining, surface micro-machining, soft lithography, x-ray lithography, LIGA techniques (e.g., X-ray lithography, electroplating, and molding), injection molding, conventional silicon-based fabrication methods (e.g., inductively coupled plasma etching, wet etching, isotropic and anisotropic etching, isotropic silicon etching, anisotropic silicon etching, anisotropic GaAs etching, deep reactive ion etching, silicon isotropic etching, silicon bulk micromachining, and the like), complementary-symmetry/metal-oxide semiconductor (CMOS) technology, deep x-ray exposure techniques, and the like. See, for example, U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311. Some or all of the teachings therein may be applied to microneedle devices, their manufacture, and their use in iontophoretic applications. In some techniques, the physical characteristics of the microneedles 206 depend on, for example, the anodization conditions (e.g., current density, etching time, HF concentration, temperature, bias settings, and the like) as well as substrate properties (e.g., doping density, doping orientation, and the like).

The microneedles 206 may be sized and shaped to penetrate the outer layers of skin to increase its permeability and transdermal transport of pharmaceutical compositions, molecules, compounds, active agents, and the like. In some embodiments, the microneedles 206 are sized and shaped with an appropriate geometry and sufficient strength to insert into a biological interface 118 (e.g., the skin or mucous membrane on a subject, and the like), and thereby increase a trans-interface (e.g., transdermal) transport of pharmaceutical compositions, molecules, compounds, active agents, and the like.

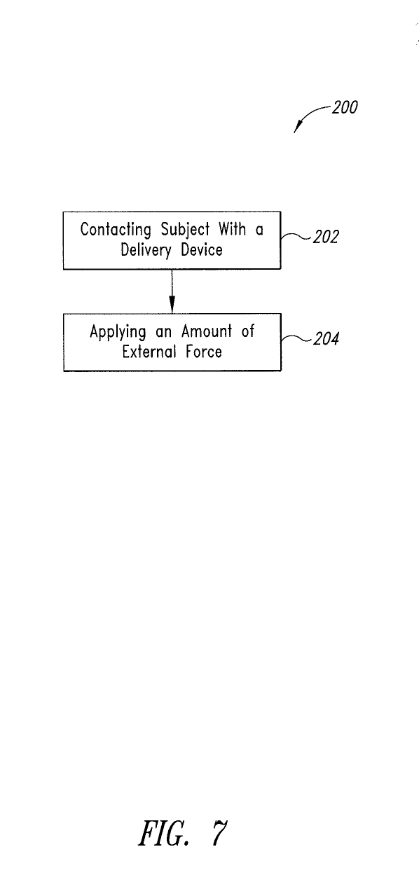

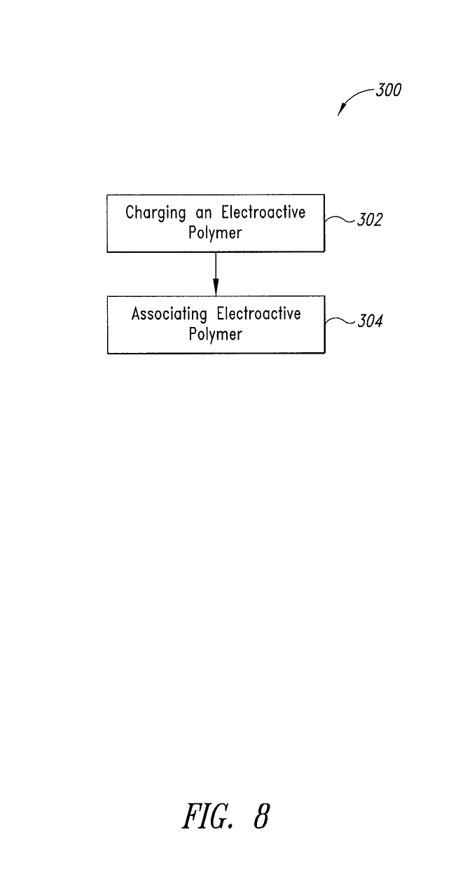

FIG. 7 shows an exemplary method 200 for treating a subject.

At 202, the method includes contacting the subject with a delivery device 10 including an effective amount of one or more active agents 36, 40, 42 and one or more membranes. In some embodiments, at least one membrane takes the form of an electroactive polymer. In some embodiments, the delivery device 10 facilitates active or passive transport of one or more active agents 36, 40, 42 to the subject. In some embodiments, the delivery device 10 facilitates active transport via iontophoretic delivery. In some embodiments, the delivery device 10 facilitates passive transport via diffusion.

At 204, the method may further include applying an amount of external force. In some embodiments, applying an amount of external force includes applying a mechanical or an electrical force.

In some embodiments, applying a mechanical or an electrical force includes applying a fixed, variable, or cyclical force. In some embodiments, the external force increases active or passive transport of one or more active agents 36, 40, 42 to the subject. In some embodiments, applying an amount of external force comprises applying a cyclical electrical force. In some embodiments, the cyclical electrical force alters the electroactive polymer from neutral to charged state. In some embodiments, the charged state is positive or negative.

In some embodiments, applying an amount of external force comprises applying a sufficient amount of external force to facilitate active or passive transport of one or more active agents to the subject. In some embodiments, applying an amount of external force comprises applying a mechanical or an electrical force sufficient to alter a charge state of the at least one membrane comprising an electroactive polymer.

FIG. 8 shows an exemplary method 300 for making an active agent delivery device.

At 302, the method 300 includes charging an electroactive polymer. In some embodiments, charging the electroactive polymer includes performing at least one of a redox reaction, an oxidation reaction, a reduction reaction, and the like.

At 304, the method 300 includes associating the electroactive polymer with at least one active agent 36, 40, 42 desired for delivery. In some embodiments, the electroactive polymer comprises polypyrrole.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to:

Japanese patent application Serial No. H03-86002, filed Mar. 27, 1991, having Japanese Publication No. H04-297277, issued on Mar. 3, 2000 as Japanese Patent No. 3040517;

Japanese patent application Serial No. 11-033076, filed Feb. 10, 1999, having Japanese Publication No. 2000-229128;

Japanese patent application Serial No. 11-033765, filed Feb. 12, 1999, having Japanese Publication No. 2000-229129;

Japanese patent application Serial No. 11-041415, filed Feb. 19, 1999, having Japanese Publication No. 2000-237326;

Japanese patent application Serial No. 11-041416, filed Feb. 19, 1999, having Japanese Publication No. 2000-237327;

Japanese patent application Serial No. 11-042752, filed Feb. 22, 1999, having Japanese Publication No. 2000-237328;

Japanese patent application Serial No. 11-042753, filed Feb. 22, 1999, having Japanese Publication No. 2000-237329;

Japanese patent application Serial No. 11-099008, filed Apr. 6, 1999, having Japanese Publication No. 2000-288098;

Japanese patent application Serial No. 11-099009, filed Apr. 6, 1999, having Japanese Publication No. 2000-288097;

PCT patent application WO 2002JP4696, filed May 15, 2002, having PCT Publication No WO03037425;

U.S. patent application Ser. No. 10/488970, filed Aug. 24, 2004;

Japanese patent application 2004/317317, filed Oct. 29, 2004;

U.S. provisional patent application Ser. No. 60/627,952, filed Nov. 16, 2004;

Japanese patent application Serial No. 2004-347814, filed Nov. 30, 2004;

Japanese patent application Serial No. 2004-357313, filed Dec. 9, 2004;

Japanese patent application Serial No. 2005-027748, filed Feb. 3, 2005; and

Japanese patent application Serial No. 2005-081220, filed Mar. 22, 2005.

As one of skill in the art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. A delivery device for providing transdermal delivery of one or more therapeutic active agents to a biological interface of a subject, comprising:
one or more active agents, and
one or more membranes, at least one membrane of the one or more membranes comprising an electroactive polymer.

2. The delivery device of claim 1 wherein the electroactive polymer is selected from the group consisting of: poly(sulfur nitride); polyacetylene; poly(ethylenedioxythiophene); poly(p-phenylene); poly(p-phenylenevinylene); poly-1,6 heptadiyne; polyphenylene sulfide; poly-m-phenylene; polyaniline; polypyrrole; polythiophene; polyisoprene; polyfuran; polyimides; polythiophenes; ionomeric polymer metal composites, carbon nanotubes, ferroelectric polymers, ionic polymer gels, vinyl copolymers; odd-numbered nylons; poly(vinylidene fluoride); polythioureas; polyacrylonitrile; poly(vinylidenecyanide vinylacetate); polyphenylethernitrile; poly(1-copolymers); and polyvinylchloride.

3. The delivery device of claim 1 wherein the electroactive polymer comprises polypyrrole.

4. The delivery device of claim 1 wherein the electroactive polymer has a net positive charge.

5. The delivery device of claim 1 wherein the electroactive polymer has a net negative charge.

6. The delivery device of claim 1, further comprising: a substrate saturated with the active agent.

7. The delivery device of claim 6 wherein the substrate comprises an absorbent layer on or in the device.

8. The delivery device of claim 1, further comprising: a medical backing.

9. The delivery device of claim 1, further comprising: an adhesive.

10. The delivery device of claim 1 wherein the device takes the form of a patch.

11. The delivery device of claim 1, further comprising: an iontophoretic component.

12. The delivery device of claim 1 wherein at least one membrane of the one or more membranes deforms in the presence of an electrical or mechanical force.

13. The delivery device of claim 1 wherein one or more active agents is transported in the presence of an electrical or mechanical force.

14. The delivery device of claim 13 wherein the electrical or mechanical force comprises a fixed, variable or cyclical force.

15. The delivery device of claim 1 further comprising one or more reservoirs.

16. The delivery device of claim 15 wherein the one or more reservoirs are bound by one or more membranes comprising an electroactive polymer.

17. The delivery device of claim 15, further comprising: two or more reservoirs.

18. The delivery device of claim 17 wherein the two or more reservoirs are bound by one or more membranes comprising an electroactive polymer.

19. The delivery device of claim 17 wherein two or more reservoirs comprise electrolyte solutions of differing compositions.

20. A method for treating a subject, comprising:
contacting the subject with a delivery device for providing transdermal delivery of one or more active agents to the subject, the delivery device comprising
a therapeutically effective amount of one or more active agents, and
one or more membranes, wherein at least one membrane comprises an electroactive polymer.

21. The method of claim 20 wherein the delivery device facilitates active or passive transport of one or more active agents to the subject.

22. The method of claim 21 wherein the active transport comprises iontophoretic delivery.

23. The method of claim 21 wherein the passive transport comprises diffusion.

24. The method of claim 20, further comprising: applying a sufficient amount of external force to facilitate active or passive transport of one or more active agents to the subject.

25. The method of claim 24 wherein applying an amount of external force comprises applying a mechanical or an electrical force sufficient to alter a charge state of the at least one membrane comprising an electroactive polymer.

26. The method of claim 25 wherein applying a mechanical or an electrical force comprises applying a fixed, variable or cyclical force.

27. The method of claim 24 wherein the external force increases active or passive transport of one or more active agents to the subject.

28. The method of claim 24 wherein applying an amount of external force comprises applying a cyclical electrical force.

29. The method of claim 28 wherein the cyclical electrical force alters the electroactive polymer from neutral to charged state.

30. The method of claim 29 wherein the charged state is positive or negative.

31. A method for making an active agent delivery device comprising:
charging an electroactive polymer, and
associating the electroactive polymer with at least one active agent desired for delivery.

32. The method of claim 31 wherein charging the electroactive polymer comprises performing a redox reaction.

33. The method of claim 31 wherein charging the electroactive polymer comprises performing an oxidation reaction.

34. The method of claim 31 wherein charging the electroactive polymer comprises performing a reduction reaction.

35. The method of claim 31 wherein the electroactive polymer comprises polypyrrole.
